(19) United States
(12) Patent Application Publication
(10) Pub. No.: US 2006/0217691 A1
(43) Pub. Date: Sep. 28, 2006

(54) Real-time therapeutic dosimetry based on dynamic response of treated tissue

(76) Inventors: Georg Schuele, Menlo Park, CA (US); Fannl Moinar, Gondelfingen (DE); Daniel V. Palanker, Sunnyvale, CA (US)

Correspondence Address: LUMEN INTELLECTUAL PROPERTY SERVICES, INC., 2345 YALE STREET, 2ND FLOOR, PALO ALTO, CA, 94306, US

(21) Appl. No.: 11/361,890

(22) Filed: Feb. 23, 2006

Related U.S. Application Data
(60) Provisional application No. 60/676,600, filed on Apr. 28, 2005.
(60) Provisional application No. 60/656,765, filed on Feb. 25, 2005.
(60) Provisional application No. 60/656,611, filed on Feb. 25, 2005.

(51) Int. Cl. A61N 5/06; A61B 18/18

(52) U.S. Cl. 606/12; 606/4; 607/88

(57) ABSTRACT

Improved optical therapy is provided. In a first aspect, improved dosimetry is provided by the use of spectrally resolved tissue reflectance as a real-time dosimetry signal. Spectrally resolving the reflectance substantially improves the sensitivity for dosimetry. An increase of spectrally resolved tissue reflectance (relative to a pre-treatment baseline) is indicative of a reversible tissue response to therapy, while a decrease of spectrally resolved tissue reflectance is indicative of approach to a threshold for irreversible tissue damage. In a second aspect, improved temperature uniformity within laser treated tissue is provided by using a treatment beam having an on-axis beam intensity substantially less than an off-axis beam intensity. The combined effects of heat flow within the treated tissue and illumination with such a beam profile can provide improved temperature uniformity compared to illumination with a conventional “top-hat” beam profile.

### CROSS REFERENCE TO RELATED APPLICATIONS

This application claims the benefit of U.S. provisional application 60/676,600, filed on Apr. 28, 2005, entitled “Real-Time Therapeutic Dosimetry based on Dynamic Response of Treated Tissue”, and hereby incorporated by reference in its entirety. This application also claims the benefit of U.S. provisional application 60/656,765, filed on Feb. 25, 2005, entitled “Optimization of the Therapy and Real-Time Dosimetry for Retinal Laser Treatment”, and hereby incorporated by reference in its entirety. This application also claims the benefit of U.S. provisional application 60/656,611, filed on Feb. 25, 2005, entitled “Method of Real-Time Therapeutic Dosimetry based on Vaso-Dynamic Response of Treated Tissue”, and hereby incorporated by reference in its entirety.

### GOVERNMENT SPONSORSHIP

This invention was made with Government support under contract number F9550-04-1-0075 from the Air Force Office of Scientific Research. The Government has certain rights in this invention.

### FIELD OF THE INVENTION

This invention relates to therapeutic treatment of tissue with optical radiation.

### BACKGROUND

Retinal treatment is one of the most common applications of lasers in medicine. Established therapies include photocoagulation and more recent developments include photodynamic therapy (PDT) and transpupillary thermo therapy (TTT). In many cases, the laser-induced effects on the retina are not directly and immediately observable. In such cases, these therapies are often performed in a “blind” fashion, without real-time feedback from the treated tissue, and dosimetry is based on animal studies or retrospective analysis of human data.

There is a strong demand for directly monitoring treatment induced effects during therapy, in order to account for patient to patient response variability. Such variability is significant, and depends on many factors, such as ocular transparency, choroidal blood perfusion, retinal light absorption, dye concentration and oxygenation level. The standard treatment protocol for PDT requires multiple treatment sessions, since effective real-time dosimetry for this treatment modality is not presently available. Since each PDT session is costly and time-consuming, the requirement for multiple sessions is a significant problem which can be alleviated by real-time dosimetry. In TTT, the thermal stress induced in the retina is a critical factor, and the therapeutic window is very narrow: below a certain threshold, there is no therapeutic effect, and a few degrees above the threshold, there is irreversible damage that can result in severe loss of vision. Here also, the benefit of real-time dosimetry is clear. Other treatments (e.g., removal of port wine stains in dermatology) would also benefit from real-time dosimetry.

Various approaches for dosimetry have been considered in the art. U.S. Pat. No. 6,733,490 considers retinal dosimetry using neural signals from the tissue being treated. U.S. Pat. No. 4,644,948 considers retinal dosimetry based on detection of a minimum of a fluorescence signal from the retina, where the fluorescence is induced by the treatment beam. U.S. Pat. No. 6,585,722 considers retinal dosimetry based on automated analysis of images of treated parts of the retina. U.S. Pat. No. 6,671,043 considers retinal photocoagulation dosimetry based on an acousto-optic signal. U.S. Pat. No. 4,758,081 considers control of retinal photocoagulation with a Raman signal. US 2004/0039378 considers dosimetry by detection of microcavitation in treated tissue.

Several investigators have considered the use of a tissue reflectance signal for dosimetry. U.S. Pat. No. 4,880,001 relates to controlling photocoagulation based on reflectance measurements at He—Ne and/or Argon ion laser wavelengths during treatment. Here the Ar ion laser beam is a treatment beam, and the He—Ne laser beam is a probe beam. U.S. Pat. No. 5,531,740 relates to automatic color activated laser therapy for dermatology. In this work, pre-existing color patterns are detected in the reflected light, and laser therapy is applied only to regions having a predetermined color (e.g., the blue of malformed veins). U.S. Pat. No. 6,540,391 related to interferometric reflectivity performed during treatment for dosimetry.

Also known in the art are methods for performing in vivo optical reflectance spectroscopy in a non-therapeutic setting for various diagnostics, as in US 2002/0151774.

However, none of the above-mentioned dosimetry approaches has found widespread acceptance (e.g., as indicated above, present-day standard treatment protocols do not rely on real-time dosimetry). Accordingly, provision of real-time dosimetry having enhanced practical utility would be an advance in the art.

### SUMMARY

According to an embodiment of the invention, improved dosimetry is provided by using spectrally resolved tissue reflectance as a sensitive measure of tissue response to laser therapy. A polychromatic probe beam is incident on tissue, and probe light reflected from a region of the tissue is detected. Spectral resolution can be provided by use of an optical filter at the probe source or detector, or can be provided directly by source or detector or by use of a spectrometer at the detector. The analyzed region of tissue can be directly illuminated by a treatment beam, or can be near a part of the tissue being illuminated by the treatment beam.

Many subtle tissue response effects are invisible to the naked eye because: (i) the eye accommodates to slow changes and by that obscures the image information; (ii) spectrally narrow changes cannot be perceived by the eye on a background of a spectrally broad image due to the low relative contribution of such change; and/or (iii) small changes can be below the dynamic range of sensitivity of the human perception. The present invention overcomes these problems and allows for direct imaging of normally invisible effects in tissue during therapy. To this end a method and apparatus are provided for monitoring and optimizing the therapeutic effect in tissue by spectrally-resolved imaging and analysis of the tissue response to the therapy.

In a preferred embodiment, this is accomplished by:

Imaging tissue in a specific spectral range (imaging either through filters or by illumination with two or more specific wavelengths);

Comparing (e.g., ratioing or subtracting) the image taken during the therapy with a baseline image taken prior to the therapy;

Using increased backscattering/reflection as a general sign of reversible tissue reaction to therapy; and/or

Using reduced backscattering/reflection as an early sign of approach to a threshold for irreversible issue damage.

Many variations are possible, including:

1) The use of crossed polarization at the probe source and detector to image deeper tissue layers;

2) The use of parallel polarization at the probe source and detector to image superficial tissue layers;

3) The use of image tracking (active/hardware and passive/software) or eye immobilization to ensure a correct spatial overlapping of the image frames or use of an image sensor with automatic image stabilization;

4) Normalization of the image brightness and/or the use of other color as reference for brightness. One could use different channels of color cameras for different tasks. One could also use a multi chip camera;

5) Analyzing the fundus reflectance in the exposed area and a ring shape around it. Ratioing the different areas of the images to each other;

6) Displaying the evaluated changes in reflectivity in color enhanced (color-coded) fashion;

7) Displaying the baseline image and actual image intermittently at a high repetition rate;

8) Overlaying these two images in an eye piece split display.

9) Ramping up the power on the same spot and tracking the tissue response. One could control the laser intensity or the laser duration responsive to the measured spectrally resolved tissue reflectance;

10) The use of analyzed spectrally resolved tissue reflectance data for displaying warning signs, providing automatic responses of the treatment system and/or dosimetry control.

Analysis of the vasodynamic and other tissue reactions to physiological stress can be used for controlling the laser parameters and duration of the treatment and/or enables a device to produce an indicative output for a physician administering the treatment for a real-time dosimetry. The output device can produce a variety of different outputs including but not limited to an output through a computer, a head-mounted display or an audible output. The invention is applicable to any laser therapy.

Another aspect of the invention relates to improving temperature uniformity tissue during laser therapy. In conventional laser thermal treatments, the highest tissue temperature is reached in the center of the treatment spot. When the center of the laser spot coincides with the foveola, the highest thermal stress is applied to this area. Due to the increased temperature, this area is at the highest risk of thermal damage. There is a very narrow therapeutic window between onset of the HSP expression (about 85% of the damage threshold) and thermal denaturation of the retina. Expression of the heat shock proteins (HSP) plays a very important role in the TTT. For an effective and efficient thermal therapy over a large area, the tissue temperature should be very uniform.

The present invention also provides a method and system for optimizing the laser thermal therapy of the retina. A treatment beam having a beam profile with an on-axis beam intensity substantially less than an off-axis beam intensity is employed to alleviate this central hot spot problem. In a preferred embodiment, a specially-designed radial intensity profile of the laser beam provides an optimized radial distribution of the laser irradiance and produces nearly constant temperature over a wide diameter range on the retina at the end of an exposure. The area where the temperature is within 85% of its maximum temperature value can be three times larger than with a typical top-hat beam profile, thereby providing for substantially improved TTT therapy.

### BRIEF DESCRIPTION OF THE DRAWINGS

FIG. 1 shows an optical radiation treatment system according to an embodiment of the invention.

FIG. 2 shows images of fundus reflectance in a 540 nm to 580 nm wavelength range during laser treatment.

FIGS. 3a-b show fundus images and spectrally resolved fundus reflectance images in a 540 nm to 580 nm wavelength range during laser treatment.

FIG. 4 shows differential images of spectrally resolved fundus reflectance at various levels of laser power.

FIG. 5a shows different areas of interest in a spectrally resolved fundus reflectance image.

FIG. 5b shows an example of temporal monitoring of a ratio of the mean gray values of the spectrally resolved reflectances of the two regions of FIG. 5a.

FIG. 6a shows a tissue temperature distribution provided by a beam having a top-hat profile.

FIG. 6b shows a tissue temperature distribution provided by a beam having an intensity that linearly decreases as the beam axis is approached.

FIG. 6c shows a tissue temperature distribution provided by a beam having a profile optimized to provide a uniform tissue temperature distribution.

FIG. 7 shows tissue temperature distributions at various times during illumination with a treatment beam having the optimized beam profile of FIG. 6c.

### DETAILED DESCRIPTION

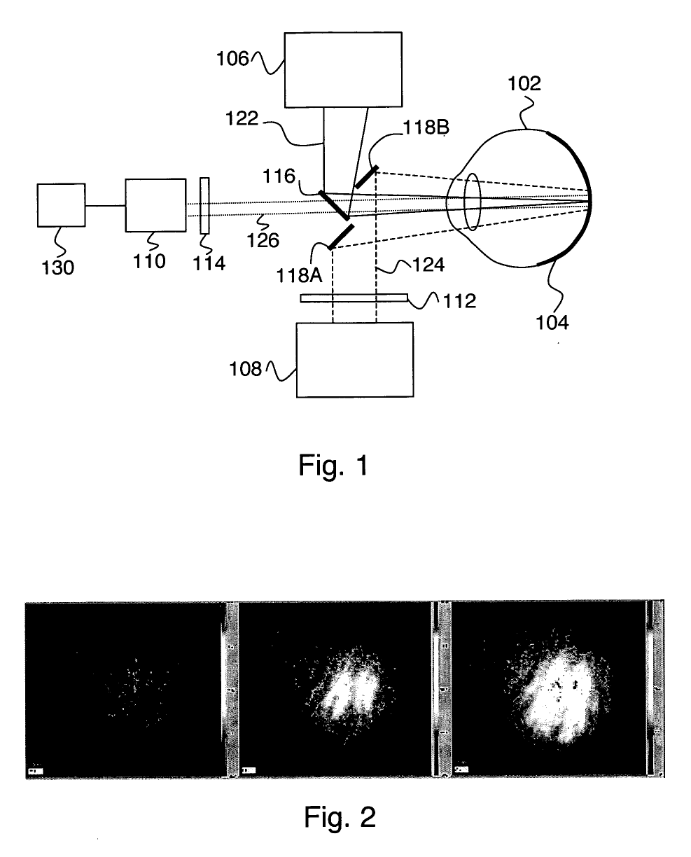

FIG. 1 shows an optical radiation treatment system according to an embodiment of the invention. A treatment optical source 106 provides a treatment beam 122 to a tissue to be treated. In this example, the tissue being treated is a retina 104 of an eye 102. A probe optical source 108 provides a polychromatic probe beam 124 to retina 104. A probe detector 110 receives reflected probe beam light 126 from a region of retina 104 at a time when the treatment beam 122 is present. For the purposes of the invention, no distinction is to be drawn between reflected light and backscattered light, since both reflection and backscattering will provide light to detector 110. Accordingly, “reflectance” in this application is understood to include both reflected light and backscattered light. A processor 130 determines a spectrally resolved tissue reflectance of retina 104 from reflected probe beam light 126. A key aspect of the present invention is the discovery that this spectrally resolved tissue reflectance is responsive to the treatment beam, and furthermore that use of a spectrally resolved reflectance significantly increases the sensitivity for dosimetry compared to prior art reflectance dosimetry approaches lacking spectral resolution. Processor 130 also includes a controller for adjusting one or more parameters of the treatment beam based on the spectrally resolved tissue reflectance. Suitable beam parameters for this adjustment include beam intensity, beam duration, beam shape and beam size. Processor 130 can be any combination of hardware and/or software suitable for implementing these functions, and can be implemented in a single unit or multiple units within the system.

For illustrative purposes, FIG. 1 shows a specific optical arrangement for providing the treatment and probe beams to the tissue being treated, and for detecting reflected probe beam light. In particular, a split mirror 118A, 118B directs the probe beam to retina 104, and a beam splitter 116 (preferably a dichroic beam splitter if the probe beam and treatment beams are at different wavelengths) directs the treatment beam to retina 104 and permits reflected probe beam light to enter detector 110. Any other optical arrangement for performing the same functions is also suitable for practicing the invention.

The spectrally resolved reflectivity is preferably provided by employing a broadband optical probe source having one or more continuous wavelength bands in its emission spectrum (e.g., a Xenon lamp, incandescent lamp, light emitting diode, gas discharge lamp, etc.), in combination with a spectral filter in detector 110. Preferably this spectral filter is a bandpass filter substantially passing a spectral range from 520 nm to 580 nm and substantially blocking probe beam light outside of this spectral range. This range is chosen to coincide with prominent absorption features in the spectrum of blood, since we have found that laser-induced vasoconstriction is a significant part of the tissue response to the treatment beam. More generally, probe beam 124 is a polychromatic beam (i.e., having two or more wavelengths). This polychromatic beam can include one or more discrete wavelengths (e.g., laser lines) and/or one or more continuous wavelength bands.

We have also found that it is preferable to include polarizers 112 and 114 in the system of FIG. 1, and to orient these polarizers such that light passed by polarizer 114 is orthogonally polarized relative to light passed by polarizer 112 (i.e., the spectrally resolved reflectance is preferably measured with crossed polarizers). The invention can also be practiced with the polarizers oriented the same way, or without any polarizers at all.

As will be considered in greater detail below, the spatial location from which reflected probe beam light is received by detector 110 may or may not be a location that is illuminated by treatment beam 122. We have found, unexpectedly, that the spectral reflectance of tissue can measurably change when nearby tissue is illuminated by a treatment beam, even though the tissue being monitored is not itself directly illuminated.

It is preferable (but not required) for the arrangement of FIG. 1 to be an imaging arrangement that provides a spatially resolved image of the spectrally resolved tissue reflectance. Conventional imaging devices (e.g., a CCD camera) can be employed as detector 110 in the system of FIG. 1 to provide such images. It is also preferred to measure a baseline spectrally resolved tissue reflectance and to directly display the changes in reflectance from the baseline induced by the treatment beam. This change is referred to as a differential or relative tissue reflectance. Imaging can be combined with baselining to provide a differential spectrally resolved reflectance image. Spectrally resolved reflectance images can be viewed or analyzed in combination with other images such as visual images of the tissue, angiography images of the tissue, and images of the treatment beam. Methods for aligning the spectral reflectance image to other images include the use of marker or fiducials, and other image alignment methods known in the art. For example, image alignment can be provided by maximizing the cross-correlation of the images being aligned.

FIG. 2 shows images of differential fundus reflectance in a 540 nm to 580 nm wavelength range during laser treatment. A baseline image taken prior to treatment laser activation was subtracted from the images taken during the laser treatment. As one can see in FIG. 2, a laser-induced vasodynamic effect in the fundus can be clearly visualized during the treatment.

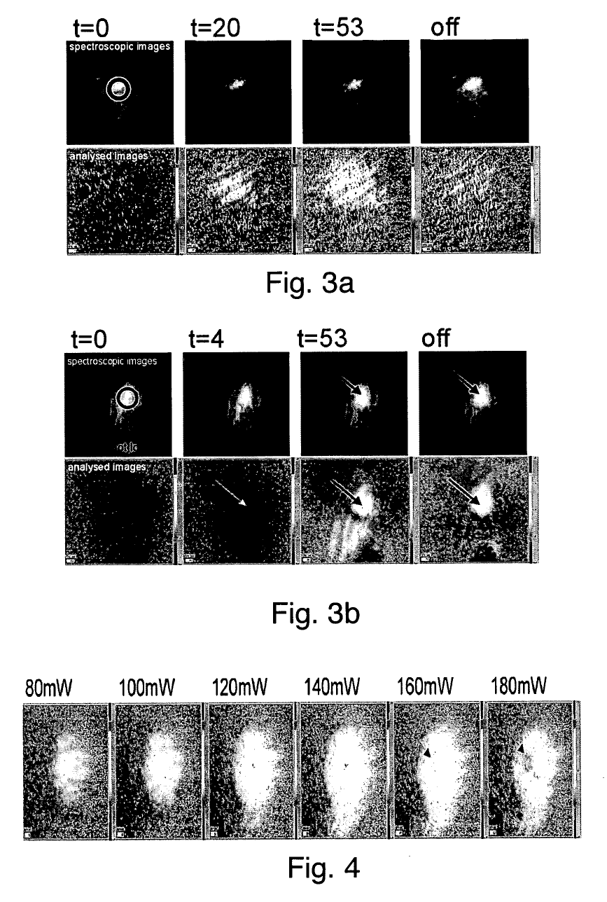

FIGS. 3a-b show fundus images and spectrally resolved fundus reflectance images in a 540 nm to 580 nm wavelength range during laser treatment. Within a few seconds of the laser treatment the reduction of fundus reflectance (as indicated by the arrows) in the exposed area indicates that a visible lesion will be created later on. Optical effects other than the above mentioned vasodynamic response can also be used for dosimetry. FIGS. 3a-b show conventional fundus images and differential spectral reflectance images for ophthalmoscopically “invisible” and visible laser spots respectively. The invisible laser spot only shows a vasoconstriction reaction indicated by the increase of fundus reflection within the spectral range of the filter. Contrary to that, a visible lesion shows a distinct reduction in fundus reflectance (indicated by arrows) in the laser spot within the first seconds of the laser pulse. Later on the fundus area around shows a vasoconstriction (see time point t=20 seconds) as indicated by the increased fundus reflection. At the end of the laser pulse a visible thermal denaturation of the retina is indicated by increased reflectance in the treatment spot (arrows). The vasoconstriction effect disappears after the laser is turned off but the denaturated lesion remains stable.

The effect that leads to the reduction of the fundus reflectance represents a tissue response to the induced stress. It is important to emphasize that the two described effects (vasodynamic response and tissue response) can be clearly differentiated since they have opposite effects on reflectance. The vasodynamic response increases the reflectance, while the tissue stress response decreases the reflectance.

FIG. 4 shows differential images of spectrally resolved fundus reflectance at various levels of laser power. In this example the treatment laser power increased every 10 seconds from 80 mW to 180 mW in steps of 20 mW. In the power range from 80 mW to 140 mW the fundus reflectance increases with power. At 160 mW a faint decrease of fundus reflection already indicates a different tissue effect, which becomes more pronounced at 180 mW. One can use such an arrangement to find an appropriate power level for a successful retinal laser treatment.

In view of these results, the following treatment method according to an embodiment of the invention is provided. The spectrally resolved tissue reflectance is monitored during irradiation by a treatment beam. An increase of spectrally resolved tissue reflectance during treatment, relative to a baseline tissue reflectance, is regarded as an indication that the treatment beam intensity is within a first intensity range characterized by reversible tissue spectral reflectance response to therapy. A decrease of spectrally resolved tissue reflectance during treatment, relative to the baseline, is regarded as an indication that a threshold for irreversible tissue damage is being approached. The adjustment of the treatment beam parameters is made in accordance with these intensity ranges. For example, if therapy is presently in the first intensity range, continue therapy at the present treatment beam power or increase treatment beam power. If therapy is presently in the second intensity range, discontinue therapy or reduce treatment beam power.

In the preceding description, “reversible” is used to indicate specifically that the changes in spectral reflectance are temporary, and that the tissue spectral reflectance returns substantially to the baseline value after completion of the therapy. A typical “reversible” change would be a vasodynamic response of the choroidal blood vessels. A vasodynamic effect, also well known as a change of “tone”, is used by different body parts to accommodate temperature effects as heating and cooling. Other characteristics of the tissue can also be changed by the therapy and these changes can persist after therapy, even though the change in spectral reflectance is reversible.

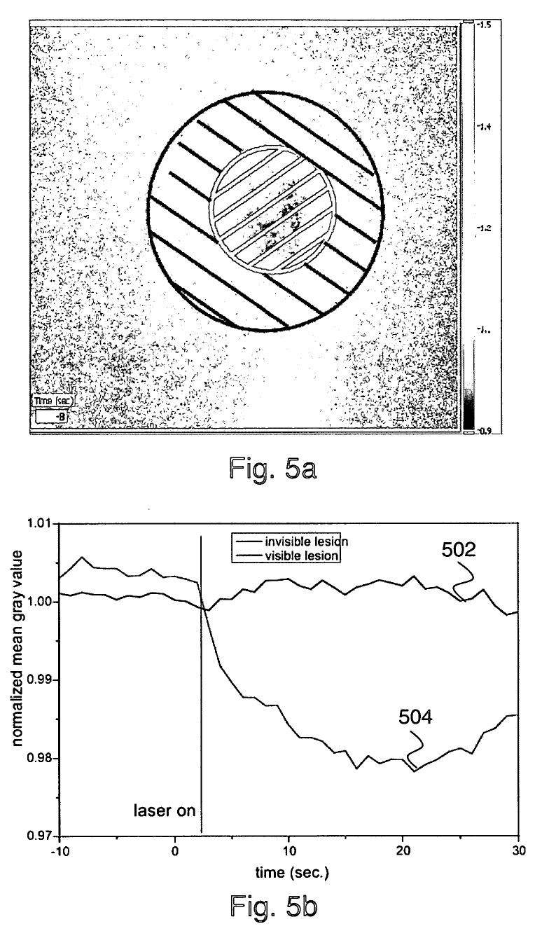

For further data analysis several regions of interest can be selected. In FIG. 5a the central disk area is used for analysis of the tissue response under the direct laser exposure, while the annular region around the disk is used for monitoring the tissue response around the laser spot.

FIG. 5b shows an example of temporal monitoring of a ratio of the mean gray values of the spectrally resolved reflectances of the two regions of FIG. 5a. For an invisible laser lesion (line 502) the mean gray value of the two areas remains the same. In case of an exposure that leads to formation of a visible lesion the area inside the treatment laser's spot (central disk of FIG. 5a) react differently from the tissue around it (line 504). Reduction of the fundus reflection in the exposed area can be used as a warning sign for a real-time laser dosimetry. To avoid formation of the visible lesions in some retinal laser therapies the laser can be turned off or the laser power can be reduced after the reflectance reduction has been detected. This effect can be detected just several seconds (˜5-10 seconds) after the laser is turned on (line 504 on FIG. 5b).

According to another aspect of the invention, the beam profile of the treatment laser beam is altered to provide a more uniform temperature distribution within tissue being treated. A Gaussian beam has an uneven optical intensity and also produces a very uneven temperature distribution within treated tissue. Accordingly, conventional laser treatment often entails application of a uniform (top-hat) irradiance in the laser spot. This is typically accomplished by imaging the output end of a multimode fiber onto the retina. However, uniform illumination does not guarantee a uniform temperature distribution, since heat will tend to escape more effectively from the edges of the illuminated region than the center, thereby leading to the formation of a central hot spot. This effect increases in significance as the duration of therapy increases. For example, during the long exposure characteristic of TTT (on the order of 60 seconds), heat spreads from the uniform source, resulting in a temperature distribution having a maximum in the center of the laser spot.

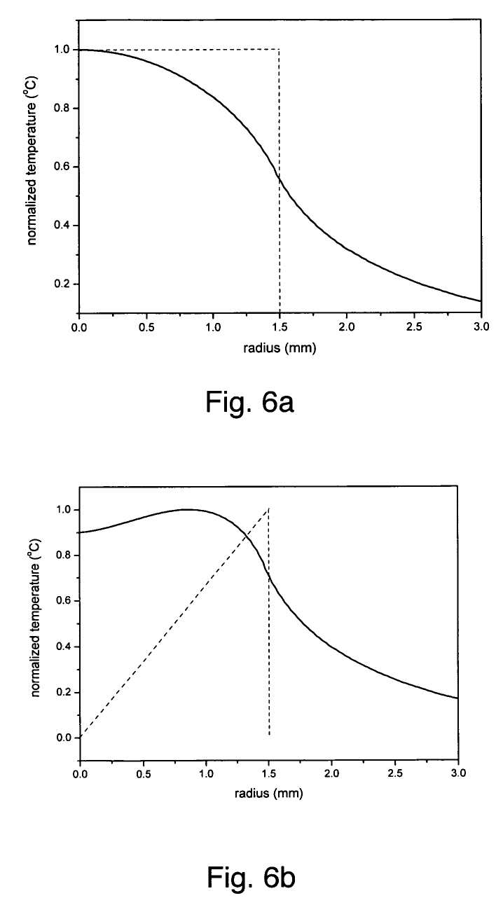

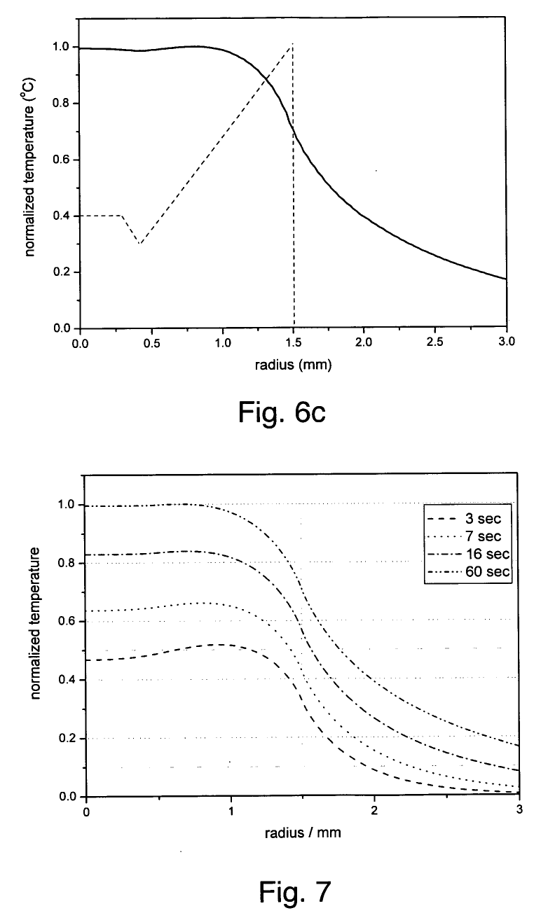

FIG. 6a shows a calculated tissue temperature distribution provided by a beam having a top-hat profile. The beam duration is 60 s, and a 3 mm diameter top-hat beam profile is assumed. The dotted line represents the radial distribution of the laser intensity. The highest temperature is reached in the center of the treatment spot. When the center of the laser spot coincides with the foveola, the highest thermal stress is applied to this area. This is highly undesirable since this area would thus be at the highest risk of damage from thermal denaturation.

According to an embodiment of the invention, the treatment beam has a beam pattern on the tissue being treated that has an on-axis intensity substantially less than an off-axis beam intensity. In this way, the tendency to form a central hot spot in the treated tissue can be reduced. Preferably, the beam pattern is rotationally symmetric about the beam axis, although this is not required.

FIG. 6b is a plot showing calculated normalized retinal temperature distribution at the end of a 60-second laser irradiation with a non-uniform laser profile of 3 mm in diameter. In this case, the laser intensity increases linearly from zero in the center to maximum at the radius of 1.5 mm, as illustrated by the dotted line. In this case, the highest temperature is not located in the center of the spot, so the foveola has a lower risk of thermal damage than the surrounding area in cases where the treatment spot is centered on the foveola.

FIG. 6c is a plot showing calculated normalized retinal temperature distribution at the end of a 60-second laser irradiation with optimized laser profile of 3 mm in diameter. In this case, the radial laser intensity is optimized to achieve a uniform radial temperature distribution. The radial laser intensity function is shown as a dashed line. In this case, the area where the temperature is within 85% of its maximum value is three times larger than with the top-hat beam profile of FIG. 6a. Such enhanced temperature uniformity is especially valuable in connection with TTT. Expression of heat shock proteins (HSP) plays a very important role in TTT. HSP expression starts roughly at the temperature rise corresponding to 85% of the thermal damage threshold. Thus, there is a very narrow therapeutic window between onset of HSP expression and thermal denaturation of the retina. For an effective and efficient thermal therapy over a large area, the tissue temperature should therefore be very uniform.

Details of the optimized beam profile will depend on details of the treatment being considered (e.g., beam size and duration) as well as on the tissue being treated (a heat flow model appropriate for the tissue being treated is necessary, and these models will vary depending on tissue type). Optimization of the beam profile for maximum temperature uniformity for a particular tissue thermal model is within the skill of an art worker, making use of the principles of the invention as described above.

FIG. 7 is a plot showing calculated normalized retinal temperature distribution for different time points during exposure with the optimized illumination of FIG. 6c. The temperature at the center of the laser beam (foveola) is lower than at the outer rim during the heating. After 60 seconds, heat diffusion from the outer parts equalizes the central temperature with that of the outer rim.

1. A method for providing optical therapy to a tissue, the method comprising:
providing a treatment beam of optical radiation to the tissue during a treatment;
providing a polychromatic probe beam of optical radiation to the tissue with a probe optical source;
receiving reflected probe beam radiation at a probe detector from a region of the tissue during the treatment;
determining a change of a spectrally resolved tissue reflectance with the probe beam during the treatment; and
adjusting one or more parameters of the treatment beam based on the spectrally resolved tissue reflectance.

2. The method of claim 1, wherein said region is illuminated by said treatment beam.

3. The method of claim 1, wherein said region is not illuminated by said treatment beam, and wherein said region is in proximity to a part of said tissue that is illuminated by said treatment beam.

4. The method of claim 1, wherein said parameters of the treatment beam are selected from the group consisting of beam intensity, beam duration, beam shape and beam size.

5. The method of claim 1, wherein said tissue is retinal tissue, wherein said region comprises a first subregion including a fundus of the retinal tissue and a second annular subregion surrounding the first region, and wherein spectral reflectances of the first and second subregions are ratioed to provide an input for said adjusting.

6. The method of claim 1, wherein said spectrally resolved tissue reflectance is measured at multiple spatially resolved locations on said tissue, thereby providing a spectral reflectance image.

7. The method of claim 6, further comprising aligning said spectral reflectance image with one or more additional images, wherein the additional images are selected from the group consisting of visual images of said tissue, angiography images of said tissue, and images of the treatment beam.

8. The method of claim 6 further comprising measuring a baseline spectral reflectance image with said probe beam and providing a display of said spectral reflectance image compared to the baseline spectral reflectance image during said treatment.

9. The method of claim 1, further comprising measuring a baseline spectral reflectance of the tissue with said probe beam when said treatment beam is not incident on said tissue;
wherein the treatment beam has a first intensity range and a second intensity range;
wherein an increase, during treatment, of said spectrally resolved tissue reflectance relative to the baseline spectral reflectance is indicative of reversible tissue spectral reflectance response to therapy in the first intensity range;
wherein a decrease, during treatment, of said spectrally resolved tissue reflectance relative to the baseline spectral reflectance is indicative of approach to a threshold for irreversible tissue damage in the second intensity range;
wherein said adjusting one or more parameters is in accordance with the first and second intensity ranges.

10. The method of claim 9, further comprising ramping up a power of said treatment beam until a decrease in said spectrally resolved tissue reflectance is observed, followed by decreasing the power of the treatment beam or terminating delivery of the treatment beam.

11. A method for providing optical therapy to a tissue, the method comprising:
providing a beam of optical radiation having a beam axis to the tissue, wherein the beam impinges on the tissue with a predetermined beam pattern, and wherein an on-axis intensity of the beam pattern is substantially less than a beam intensity at an off-axis location of the beam pattern.

12. The method of claim 11, wherein said beam pattern is substantially rotationally symmetric about said beam axis.

13. A system for providing optical therapy to a tissue, the system comprising:
a treatment optical source providing a treatment beam of optical radiation to the tissue during a treatment;
a probe optical source providing a polychromatic probe beam of optical radiation to the tissue;
a probe detector receiving reflected probe beam light from a region of the tissue during the treatment;
a processor, wherein a change of a spectrally resolved tissue reflectance is determined from the reflected probe beam light during the treatment;
a controller, wherein one or more parameters of the treatment beam is adjusted based on the spectrally resolved tissue reflectance.

14. The system of claim 13, wherein said polychromatic probe beam has a spectrum which includes zero or more discrete wavelengths and zero or more continuous wavelength bands.

15. The system of claim 13, wherein said detector is configured to receive reflected probe beam light having the same polarization as said probe beam and to substantially block reflected probe beam light having an orthogonal polarization relative to said probe beam.

16. The system of claim 13, wherein said detector is configured to receive reflected probe beam light that is orthogonally polarized relative to said probe beam and to substantially block reflected probe beam light having the same polarization as said probe beam.

17. A system for providing optical therapy to a tissue, the system comprising:
a treatment optical source providing a treatment beam of optical radiation having a beam axis to the tissue;
wherein the beam impinges on the tissue with a predetermined beam pattern,
wherein an on-axis intensity of the beam pattern is substantially less than a beam intensity at an off-axis location of the beam pattern.
